(19) United States
(12) Patent Application Publication
(10) Pub. No.: US 2012/0156307 A1
(43) Pub. Date: Jun. 21, 2012

(54) MEDICAL USES OF NANOCLUSTERED WATER

(75) Inventors: Yongge Chen, Guangzhou (CN); Roberto De Noni, Fregona (IT); Giorgio Reiner, Como (IT); Paolo Galfetti, Senna Comasco (IT)

(73) Assignee: APR Nanotechnologies s.a., Balerna (CH)

(21) Appl. No.: 13/324,702

(22) Filed: Dec. 13, 2011

Related U.S. Application Data
(60) Provisional application No. 61/423,712, filed on Dec. 16, 2010.

(51) Int. Cl. A61K 33/00; A61P 31/10; A61P 31/12; A61P 33/00; A61P 31/06; A61P 31/14; A61P 31/22; A61P 33/04; A61P 33/12; A61P 1/04; A61P 17/06; A61P 17/10; A61P 17/00; A61P 31/18; A61P 17/02; A01N 1/02; A61P 31/04

(52) U.S. Cl. 424/600; 435/1.1

(57) ABSTRACT

The present invention relates to methods of treating or preventing an infection or infectious disease in a mammal, or sanitizing mammalian tissue, or treating or preventing psoriasis in a mammal, comprising topically administering to said mammal an electrolytic acid water comprising free chlorine, wherein: a) from 90% to 99.9% of said free chlorine is present in the form of hypochlorous acid; b) said water has a pH of from 0.5 to 5.0; and c) said water has oxidation reduction potential (ORP) of greater than 1100 mV. The present invention also relates to methods of healing, repair or regeneration of a tissue in a mammal comprising topically administering to said tissue said electrolytic acid water.

### CROSS-REFERENCE TO RELATED APPLICATIONS

This application claims benefit of U.S. Provisional Application No. 61/423,712, filed Dec. 16, 2010, which is incorporated herein by reference in its entirety.

### FIELD OF THE INVENTION

The present invention relates to various medical uses of nanoclustered water, including tissue healing, repair and regeneration, as well as treatment of infections.

### BACKGROUND OF THE INVENTION

### Infection and Infectious Diseases

An infection is the detrimental colonization of a host organism by parasite species. Infecting parasites seek to utilize the host's resources to reproduce, often resulting in disease. Colloquially, infections are usually considered to be caused by microscopic organisms or microparasites like viruses, prions, bacteria, and viroids, though larger organisms like macroparasites and fungi can also infect. An infectious disease is a clinically evident illness resulting from the presence of pathogenic biological agents, including pathogenic viruses, pathogenic bacteria, fungi, protozoa, multicellular parasites, and aberrant proteins known as prions. These pathogens are able to cause disease in animals and/or plants. Infectious pathologies are also called communicable diseases or transmissible diseases due to their potential of transmission from one person or species to another by a replicating agent (as opposed to a toxin). Although various treatments for infections and infectious diseases are available, they generally are associated with a variety of drawbacks. Therefore, there is a need to develop new methods of treatment which provide better efficacy, reduced side effects, reduced cost, more convenient administration and longer shelf life of the medical product being administered.

### Sinus Infection

Sinus infection, or sinusitis, is an inflammation of the paranasal sinuses. Sinusitis is one of the most common clinical pictures presented in the population. Approximately 15% of the population of the western industrialized nations suffers from chronic inflammation of the paranasal sinuses. Those affected almost always feel unwell. Typical symptoms include headache, cough, fever, restricted breathing through the nose, and an impaired sense of smell and taste. Sinusitis often develops from rhinitis, when the discharge of secretions from the paranasal sinuses is obstructed by swelling of the mucous membranes or by anatomical circumstances. The resulting accumulation of secretions represents an ideal breeding ground for microorganisms. Sinusitis is in most cases triggered by viruses, for example rhinoviruses, adenoviruses or RS viruses. An impaired immune defense then often leads to a secondary bacterial infection or what is referred to as a bacterial superinfection. The bacterial pathogens are in most cases Haemophilus influenzae and Streptococcus pneumoniae.

Sinusitis may be either acute or chronic. Acute sinusitis is frequently subsequent to a cold and follows a relatively brief course of between several days and three weeks. Acute sinusitis may be caused by viral or bacterial infection of the nose, the throat, and the upper respiratory tract. Chronic sinusitis is defined as sinusitis lasting for more than 30 days. Bouts are frequent throughout the year. Symptoms of chronic sinusitis include runny nose, congestion, headaches, and diminished sense of smell. In addition to being of longer duration than acute sinusitis, chronic sinusitis is more difficult to treat with conventional decongestants and antibiotics, which are the medications of choice for acute sinusitis.

Various therapeutic measures (e.g., nose drop, nasal sprays, antibiotics, and surgical procedures) are presently available for the treatment of sinusitis. The main aim of therapy is to reduce the inflammation as far as possible and restore the natural mucosal discharge of the paranasal sinuses. There still remains a need to develop a method of treating sinusitis with better efficacy, reduced side effects, convenient administration and longer shelf life of the medical product being administered.

### Psoriasis

Psoriasis is a chronic autoimmune disease that appears on the skin. It occurs when the immune system sends out faulty signals that speed up the growth cycle of skin cells. Psoriasis is not contagious. There are five types of psoriasis: plaque, guttate, inverse, pustular and erythrodermic. The most common form, plaque psoriasis, is commonly seen as red and white hues of scaly patches appearing on the top first layer of the epidermis (skin). Some patients, though, have no dermatological symptoms. In plaque psoriasis, skin rapidly accumulates at these sites, which gives it a silvery-white appearance. Plaques frequently occur on the skin of the elbows and knees, but can affect any area, including the scalp, palms of hands and soles of feet, and genitals. In contrast to eczema, psoriasis is more likely to be found on the outer side of the joint.

The disorder is a chronic recurring condition that varies in severity from minor localized patches to complete body coverage. Fingernails and toenails are frequently affected (psoriatic nail dystrophy) and can be seen as an isolated symptom. Psoriasis can also cause inflammation of the joints, which is known as psoriatic arthritis. Ten to fifteen percent of people with psoriasis have psoriatic arthritis. The cause of psoriasis is not fully understood, but it is believed to have a genetic component, and local psoriatic changes can be triggered by an injury to the skin known as the Koebner phenomenon. Various environmental factors have been suggested as aggravating to psoriasis, including stress, withdrawal of systemic corticosteroid, as well as other environmental factors, but few have shown statistical significance. There are many treatments available, but because of its chronic recurrent nature, psoriasis is a challenge to treat. Therefore, there is a need to develop better methods of treatment.

### Tissue Healing, Repair and Regeneration

Tissue healing refers to the body's replacement of destroyed tissue by living tissue and comprises two essential components—regeneration and repair. The differentiation between the two is based upon the resultant tissue. In regeneration, specialized tissue is replaced by the proliferation of surrounding undamaged specialized cells. In repair, lost tissue is replaced by granulation tissue which matures to form scar tissue. Wound is an example of an injured tissue. Wound healing is a natural restorative response to tissue injury. Healing is the interaction of a complex cascade of cellular events that generates resurfacing, reconstitution, and restoration of the tensile strength of injured skin. Healing is a systematic process, traditionally explained in terms of three classic phases: inflammation, proliferation, and maturation. A clot forms and inflammatory cells debride injured tissue during the inflammatory phase. Epithelialization, fibroplasia, and angiogenesis occur during the proliferative phase. Meanwhile, granulation tissue forms and the wound begins to contract. Finally, during the maturation phase, collagen forms tight cross-links to other collagen and with protein molecules, increasing the tensile strength of the scar. There is still a need to develop better methods of healing, repair or regeneration of tissues in a mammal.

### Sanitizing, Hydrating and Storing Mammalian Tissues or Organs

Sanitizing, hydrating and storing a mammalian tissue or organ externalized from the body of said mammal, which is preserved for implantation or transplantation, has been a challenging task in the medical field. Specifically, tissues for human or animal implantation are preserved, after being explanted from a donor and while waiting for implantation, in appropriately provided sterile banks, usually after dehydration (for example by freeze drying), so as to slow and prevent the growth of bacteria. Then, prior to implantation, tissues needs to be rehydrated and sanitized. Therefore, there remains a need to develop methods of sanitizing, hydrating and storing the tissues which offer advantages, such as easy handling, less chance of infection, less process time and cost effective.

### High ORP Acid Water

It is known that aqueous solutions of salts, particularly sodium chloride, as a consequence of an electrolytic treatment, are split into two liquid products, one having basic and reducing characteristics (generally known as cathode water or alkaline water) and another (generally known as anode water or acid water) having acid and oxidizing characteristics.

Conventional electrolytic waters suffer the acknowledged drawback of having very limited preservation. A few days after preparation, the product in fact generally tends to degrade and lose its properties. Known electrolytic waters, therefore, must be prepared and used substantially on the spot. Accordingly, the commercial utilization of the product in itself is extremely disadvantageous, since the shelf life of any ready-made packages is dramatically limited.

The stability of an electrolyzed oxidizing water is reported in the article “Effects of Storage Conditions and pH on Chlorine Loss in Electrolyzed Oxidizing (EO) Water”—Journal of Agricultural and Food Chemistry—2002, 50, 209-212 by Soo-Voon Len, et al. In Soo-Voo Len, electrolyzed water with an acidic pH (2.5-2.6), high ORP (1020-1120 mV), and a free chlorine content of ˜50 ppm (53-56 ppm) was generated using a current intensity of 14 Ampere and 7.4 Volt. Unfortunately, in an open condition at 25° C., the chlorine in the electrolyzed water was completely lost after 30 hours when agitated, and after 100 hours when not agitated. Furthermore, in a closed dark condition at 25° C., the free chlorine in the electrolyzed water decreased by approximately 40% after 1400 hours (about 2 months).

The stability of electrolyzed oxidizing water also is reported in the article “Effects of storage conditions on chemical and physical properties of electrolyzed oxidizing water”—Journal of Food Engineering 65 (2004) 465-471 by Shun-Yao Hsu, et al. In Shun-Yao Hsu, the electrolyzed water of “formulation J” had an acidic pH (2.61), high OPR (1147 mV), and a free chlorine content of 56 ppm. The article reports that in a closed condition at 25-30° C., the free chlorine in the electrolyzed water was 43 ppm after 21 days, a 23% loss.

Thus, there remains a need for acidic electrolytic water with a greater chemical stability than traditional waters. There is a particular need for water with a greater stability during long term storage, so as to allow for the commercial utilization of acidic electrolytic water products.

### SUMMARY OF THE INVENTION

It has unexpectedly been discovered that the acidic nanoclustered water, characterized by a high oxidation reduction potential and a particular composition of chlorine species, among other characteristics such as pH, ORP and NMR half line width, is particularly useful for a variety of medical applications, e.g., treating or preventing an infection or infectious disease or psoriasis in a mammal; healing, repair or regeneration of a tissue in a mammal; and washing, sanitizing, hydrating or storing a mammalian tissue or organ externalized from the body of said mammal. The methods disclosed in the present application offer various advantages over the existing available methods, e.g., better efficacy, reduced side effects, easier handling, more convenient and flexible administration and less cost.

It also has unexpectedly been discovered that acidic nanoclustered water having a particular composition of chlorine species has a greater chemical stability than traditional waters. The unique composition can result from particular membrane and electrodes used in the electrolyzing equipment, which can produce a high current intensity without causing the electrodes to break up on their surface and release heavy metals that may adversely affect stability.

Additional embodiments and advantages of the invention will be set forth in part in the description which follows, and in part will be obvious from the description, or may be learned by practice of the invention. The embodiments and advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a schematic view of an electrolytic device comprising an electrolysis chamber and two electrodes.

FIG. 2 is side cross-view of a human eye, depicting the various components of the eye.

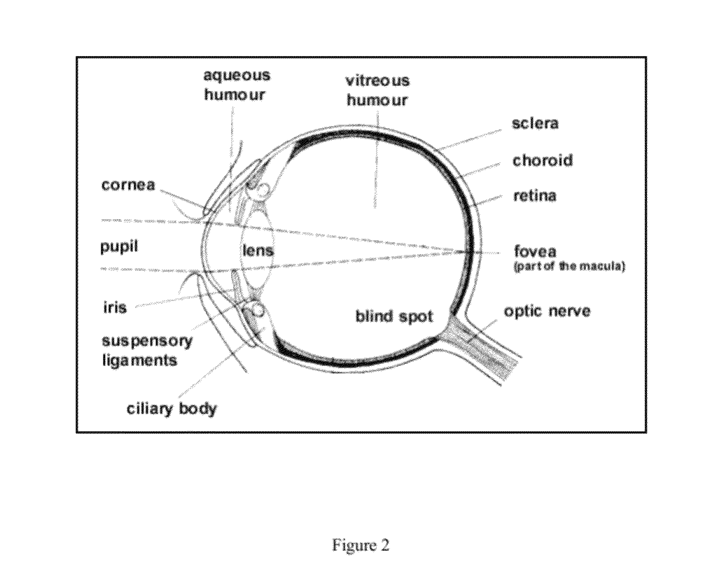

FIG. 3 is a graph illustrating the modulation of the immune system cytokines by Acidic Nanoclustered Water.

FIG. 4 is a set of graphs illustrating the concentration of various chlorine species as a function of pH.

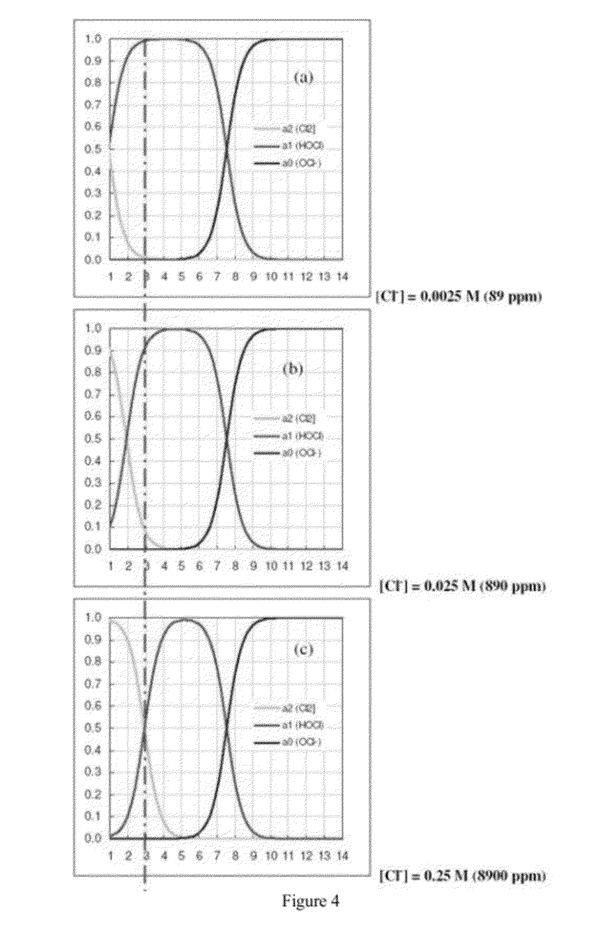

FIG. 5 is a graph illustrating the loss of free chlorine in high and low chloride Acidic Nanoclustered Water in an open, agitated, exposed condition over 24 hours.

### DETAILED DESCRIPTION OF THE INVENTION

The present invention may be understood more readily by reference to the following definitions and detailed description of preferred embodiments of the invention and the non-limiting Examples included therein.

### DEFINITIONS AND USE OF TERMS

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

As used herein, the term “fluid” is used to reference any pure fluid, solution or suspension which is capable of producing a non-spontaneous chemical reaction if subjected to electrolysis. One highly preferred fluid is water. The term “water” is used to reference any type of water, such as tap water, filtered water, deionized water, and distilled water. Once subjected to electrolysis, the water separates into two liquid fractions, which for the sake of simplicity are referenced here as acid water or anode water and as cathode water or alkaline water.

The expression “stability over time” is used to mean that the acid water, if kept sheltered from the light, air and heat, keeps its chemical and physical properties, particularly its pH, ORP and/or NMR half line width, substantially unchanged for greater than 60 or 90 days, preferably greater than 180 days, even more preferably greater than 365 days, up to two, three or even five years. By substantially unchanged, it is meant that the property under evaluation does not vary by more than 50, 30, 15, 10, 5, or even 3% during the applicable time frame.

The term “high ORP water” refers to water having an oxidation reduction potential greater that +600. The ORP preferably ranges from +600 to +1350 mV, more preferably from +800, +900, or +1000 mV to +1300 mV, most preferably from +1000 to +1250 mV.

The term “acid water” or “acidic water” refers to water having a pH less than 7.0. The pH of the acid water preferably ranges from 0.5, 1.0 or 2.0 to 6.5, 6.0, 5.0, 4.0, or 3.0, and most preferably ranges from 1.0 to 4.0.

The term “electrolytic water,” when used herein, means water produced by the process of electrolysis, and is preferably characterized by an oxide reduction potential (ORP) and/or pH that reflects its acid or alkaline nature.

As used herein, “therapeutically effective amount” refers to an amount sufficient to elicit the desired biological response. The therapeutically effective amount or dose can depend on the age, sex and weight of the patient, and the current medical condition of the patient. The skilled artisan will be able to determine appropriate dosages depending on these and other factors in addition to the present disclosure.

The terms “treating” and “treatment,” when used herein, refer to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

When the context allows, the term “significantly” can be interpreted to mean a level of statistical significance, in addition to “substantially.” The level of statistical significance can be, for example, of at least p<0.05, of at least p<0.01, of at least p<0.005, or of at least p<0.001. When a measurable result or effect is expressed or identified herein, it will be understood that the result or effect can be evaluated based upon its statistical significance relative to a baseline.

The term “topical” or “topically” can be interpreted to mean any application to exposed surfaces of a human or animal body, i.e., skin, mucosa, sinuses, eye, nose, ear, ear canal, mouth, lips, tongue, nails, genitalia or vagina.

The term “skin” can be interpreted to mean membranous tissue forming the external covering or integument of a human or an animal and consisting in vertebrates of the epidermis and dermis.

The terms “sanitize”, “sanitization” or “sanitizing” in the invention reference the provision of a combined effect of disinfection, sanitization and cleaning. In particular, the disinfection effect comprises a bactericidal, fungicide, sporicidal and virucidial effect.

Throughout the description and claims of this specification, the word “comprise” and variations of the word, such as “comprising” and “comprises,” means “including but not limited to,” and is not intended to exclude, for example, other additives, components, integers or steps.

As used in the specification and the appended claims, the singular forms “a,” “an” and “the” or like terms include plural referents unless the context clearly dictates otherwise. Thus, for example, reference to “an ingredient” includes mixtures of two or more ingredients, and the like. The word “or” or like terms as used herein means any one member of a particular list and also includes any combination of members of that list.

### Methods

In one embodiment, the invention provides methods of treating or preventing lesions in the tissues or organs of a mammalian subject, especially the skin, mucosal tissues, eyes, nose, ears, sinuses, ear canal, mouth, lips, tongue, nails, knees, scalp, palms of hands, soles of feet, genitalia and vagina, by contacting said tissue or organ with an electrolytic acid water of the present invention. Lesions treatable or preventable by the methods of this invention include any localized pathological change in the bodily organ or tissue, and include acute wounds, chronic wounds, lacerations, scrapes, burns, surgical incisions, and ulcers (venous statis, neurotrophic/diabetic, and arterial/ischemic ulcers). In one particular embodiment, the methods are used to cleanse the site of a surgical incision prior to suturing. In other embodiments, the methods are used to treat apthous ulcers or diabetic ulcers.

In another embodiment, the invention provides methods of treating or preventing infections in the tissues or organs of a mammalian subject; especially the skin, mucosal tissues, eyes, nose, ears, sinuses, ear canal, mouth, lips, tongue, nails, knees, scalp, palms of hands, soles of feet, genitalia and vagina, by contacting said tissue or organ with an electrolytic acid water of the present invention. Any mucosal tissue can be treated, especially the mucosal membranes that line the inner surfaces of the digestive and respiratory organs, the urogenital system, the accessory sinuses of the nose, the middle ear, and the excretory ducts of glands.

In one embodiment the infection or infectious disease is a bacterial infection such as Anthrax, Bacterial meningitis, Botulism, Brucellosis, Campylobacteriosis, Cat scratch disease, Cholera, Epidemic Typhus, Gonorrhea, Impetigo, Leprosy, Legionellosis, Listeriosis, Lyme disease, Melioidosis, MRSA infection, Nocardiosis, Pertussis, Plague, Pneumococcal pneumonia, Psittacosis, Q fever, Rocky Mountain Spotted Fever, Salmonellosis, Scarlet fever, Shigellosis, Syphilis, Tetanus, Trachoma, Tuberculosis, Tularemia, Typhoid fever, Typhus and Urinary tract infections, Stafilococcus Aureus, Pseudomonans aeruginosa and Prpionobacterium acne.

In another embodiment the infection or infectious disease is a viral infection such as AIDS, AIDS related complex, Chickenpox, Common cold, Cytomegalovirus infection, Colorado tick fever, Ebola haemorrhagic fever, Dengue fever, Hand, foot and mouth disease, Hepatitis, Herpes simplex, Herpes zoster, HPV, Influenza, Lassa fever, Measles, Marburg haemorrhagic, fever, Infectious mononucleosis, Mumps, Poliomyelitis, Progressive multifocal leukencephalopathy, Rabies, Rubella, SARS, Smallpox, Viral encephalitis, Viral gastroenteritis, Viral meningitis, Viral pneumonia, West Nile disease and Yellow fever.

In still another embodiment the infection or infectious disease is a fungal infection such as Aspergillosis, Blastomycosis, Candidiasis, Coccidioidomycosis, Cryptococcosis, Histoplasmosis, Tinea pedis, Candida albicans, Trichophyton mentagrophytes and Trichophyton rubrum. In another embodiment, the invention is a parasitic infection selected from African trypanosomiasis, Amebiasis, Ascariasis, Babesiosis, Chagas disease, Clonorchiasis, Cryptosporidiosis, Cysticercosis, Diphyllobothriasis, Dracunculiasis, Echinococcosis, Enterobiasis, Fascioliasis, Fasciolopsiasis, Filariasis, Free-living amebic infection, Giardiasis, Gnathostomiasis, Hymenolepiasis, Isosporiasis, Leishmaniasis, Metagonimiasis, Myiasis, Onchocerciasis, Pediculosis, Scabies, Schistosomiasis, Taeniasis, Toxocariasis, Toxoplasmosis, Trichinellosis, Trichuriasis, Trichomoniasis, Trypanosomiasis and Trypanosoma.

In still further embodiments the invention provides methods of treating particular disorders of the skin, nails or other tissues. Thus, in yet another embodiment the invention provides methods of treating skin, nail or tissue disorders by contacting, immersing, or irrigating the skin, nail or tissue with an electrolytic acid water of the present invention, wherein the disorder is selected from psoriasis onychomycosis, herpes virus, candida, T. mentagrophytes, gingivitis, aphthous ulcers, cornea ulcers, psoriasis, acne, atopic dermatitis, cellulites, folliculitis, boils, carbuncles, erysipelas, erythrasma, impetigo, paronychia, staphylococcal infection, cold sores (herpes simplex virus Type 1 and Type 2), HIV, moluscum contagiosum, chicken pox, measles, shingles, warts, candidiasis, athlete's foot (tinea pedis), jock itch (tinea cruris), ringworm (tinea corporis), face fungus (tinea faciei), tinea versicolor, fungal nail infections, fungal hair infections, lice, creeping eruption and scabies. Still other disorders include plaque, guttate, inverse, pustular or erythrodermic.

In still another embodiment, the invention is related to methods are carried out using a topical dosage form, such as a wash, a drop (i.e. eye drop, ear drop, nose drop, mouth drop), a spray (i.e. mouth sprays, nasal sprays), solutions, paste, gels, creams and ointments.

In another embodiment, the invention is related to methods of healing, repair or regeneration of a tissue in a mammal comprising topically administering to said tissue an electrolytic acid water of the present invention.

In yet another embodiment, the invention is related to methods of washing, sanitizing, hydrating or storing a mammalian tissue or organ externalized from the body of said mammal, comprising contacting said tissue or organ with, or immersing said tissue or organ in, an electrolytic acid water of the present invention. Suitable tissues and organs include, for example, hearts, kidneys, liver, lungs, pancreas, intestine, thymus, bones, tendons (musculoskeletal grafts), cornea, skin, heart valves and veins.

For all of the methods described in the above, the step of administering the water can be performed between 1 and 20 times per day using any method known in the art, but is preferably undertaken more than once during a 24 hour period. The administration can be carried out for a period of time including one week, 10 days, two weeks, three weeks, one month, or continually as a maintenance therapy.

For all of the methods described above, although the preferred administration route is topical administration, all other administration routes known in the art are suitable, e.g., orally, rectally, parenterally, ocularly, inhalationaly, or topically. The doses to be administered are determined depending upon age, body weight, symptom, the desired therapeutic effect, the route of administration, and the duration of the treatment etc. In the human adult, the doses per person per dose are generally between 0.1 mg and 500 mg, up to several times per day.

The Acid Waters

The acid waters of the present invention are preferably characterized by one or more characteristics selected from pH, ORP, conductivity, free chlorine, total chlorine chloride, chlorites, chlorates, percentage of HClO as free chlorine species, and NMR half line width. In one embodiment, the waters are characterized by all of the foregoing characteristics. In another embodiment the water are characterized by pH, ORP, free chlorine, percentage of HClO as free chlorine species, chloride, and NMR half line width. In yet another embodiment the waters are characterized by pH, ORP, free chlorine, percentage of HClO as free chlorine species, and chloride.

The ORP of the electrolytic acid water preferably ranges from +600 to +1350 mV, more preferably from +800, +900, 1000 or +1100 mV to +1300, 1250 or +1200 mV, most preferably from +1000 to +1250 mV. The pH of the acid water preferably ranges from 0.5 or 1.0 to 6.5, 6.0, 5.0, 4.0, or 3.0, and most preferably ranges from 1.0 to 4.0.

Nuclear magnetic resonance ¹⁷O NMR measures, particularly when evaluated at the half way point of the water peak, are useful to measure the quality of acid waters of the current invention, because they reflect intrinsic properties of the water structure such as the median molecular cluster size of H₂O molecules, and the distribution of molecular cluster sizes, in addition to contaminants such as ionic species within the water. In most preferred embodiments, the ¹⁷O NMR half line width for the acid water is equal to or greater than 42, 45, 46, or 47, and less than 100, 75, 60, 56, 53, 51, 50 or 49 Hz, wherein the range can be selected from any of the foregoing endpoints. Thus, for example, in preferred embodiments, the acid water of the present invention has an NMR half line width ranging from 45 to less than 51 Hz, or 45 to less than 50 Hz, or 46 to less than 50 Hz. In other preferred embodiments, the acid water of the present invention has a NMR half line width using ¹⁷O of less than about 60, 56, or 52 Hz, preferably greater than about 42 or 45 Hz.

The acid water may also be characterized by the presence and quantity of chlorine species in the water. One of the following assays or any combination of the following assays may be used to characterize the water. The percentage of HClO as a free chlorine species preferably exceed 85%, 90%, or 95%. According to the free chlorine assay (spectrophotometric method), or the total chlorine assay (spectrophotometric method), the water may be defined as containing less than 85, 70, 60, 55, 52 or even 50 mg/l of chlorine species, optionally limited by a lower bound of 20, 30 or 40 mg/l. According to the total chlorine assay (iodometric method), the water may be defined as containing less than 80, 70, 65, or even 62 mg/l of chlorine species, optionally limited by a lower bound of 20, 30 or 40 mg/l. According to the UNI 24012 (Mercurimetric method) chloride assay, the water may contain greater than 50, 100, 130, 150 or even 170 mg/l of chloride, and/or less than 250 or 200 mg/l. Chlorites (as ClO₂₋), when measured by EPA 300.1 (1997) (detection limit 100 ug/l), are preferably non-detectable. Chlorates (ClO₃₋), when measured by EPA 300.1 (1997) (detection limit 0.1 mg/l), are preferably present in an amount less than 10, 5, 2, or even 1 mg/l.

Although in certain embodiments the acid water may contain oxidizing chlorine species in amounts of up to 60 or even 100 mg/l, in a preferred embodiment the acid water according to the invention is essentially free of oxidizing chlorine species, or other anionic residues of salts that are generated during the electrolytic process, i.e. less than 10 or even 5 mg/l, and preferably undetectable.

In a particularly desirable embodiment, the water can be characterized by conductivity, the presence of dissolved chlorine gas (Cl₂), hypochlorous acid (HOCl) and chloride ions (Cl⁻), and by the presence of negligible quantities of hypochlorite ion (OCl⁻). In water, the relative amount of chlorine and hypochlorous acid is strongly affected by the amount of chlorides. Specifically, an increase in chlorides results in an increase in the amount of chlorine gas with respect to hypochlorous acid as according to the following equilibrium:

Cl⁻+H⁺+HOCl [symbol] Cl₂+H₂O

Because of the relationship between the amount of chlorides and the amounts of chlorine gas and hypochlorous acid, the amount of chloride in the water is preferably very low (less than 200 ppm) to ensure that the free chlorine in the water is almost exclusively in the form of hypochlorous acid.

The relationship between the four species Cl₂, HOCl, Cl⁻, and OCl⁻ can be understood using the disassociation equilibria of gaseous chlorine in water as described below, in which Cl₂, HOCl, and OCl⁻ are the three possible forms of free total chlorine:

Cl₂+H₂O=Cl⁻+H⁺+HOCl Kₐ₁≈3×10⁻⁴

HOCl═H⁺+OCl⁻ Kₐ₂=2.9×10⁻⁸

As can be seen in the above equations, chlorine generation occurs in the presence of an excess of Cl⁻. Furthermore, the amount of the three forms of free total chlorine as a function of pH and Cl⁻ can be determined algebraically by using the above described equilibria as follows:

αCl₂=[H⁺]²[Cl⁻]/([H⁺]²[Cl⁻]+[H⁺]Kₐ₁+Kₐ₁Kₐ₂)

αHClO=[H⁺]Kₐ₁/([H⁺]²[Cl⁻]+[H⁺]Kₐ₁+Kₐ₁Kₐ₂)

αClO⁻=Kₐ₁Kₐ₂/([H⁺]²[Cl⁻]+[H⁺]Kₐ₁+Kₐ₁Kₐ₂)

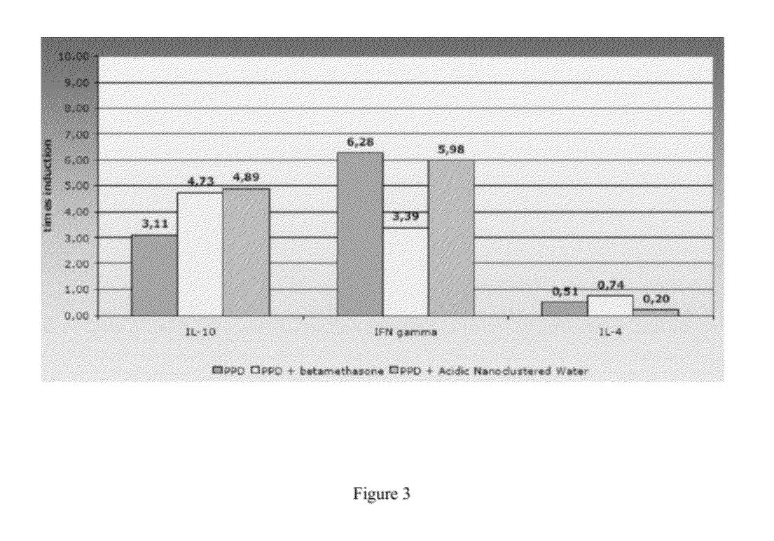

These equations can be used to simulate the chlorine concentration at different pH values. For example, FIG. 3 is a graphical simulation of the concentration of various chlorine species as a function of pH. As can be seen in FIG. 3, at a typical pH for the water of 2.8-3.0 (indicated by the green dash-and-dotted line), free chlorine is predominantly present as Cl₂ and HClO, and the relative amount of the two species is strongly affected by the amount of chlorides, and increase of which results in an increase of the amount of chlorine gas with respect to HClO.

It is generally recognized that diluted hypochlorous acid solutions are unstable due to decomposition. This decomposition can occur according to a first pathway:

2HOCl→2HCl+O₂

2HCl+2HOCl→2Cl₂+2H₂O

Or as according to a second pathway, in which chlorous acid (HClO₂) is an intermediate in the formation of chloric acid (HClO₃):

2HOCl→[HClO₂]+HCl

2HOCl+[HClO₂]→HClO₃+HCl

HClO₃+HCl+HOCl→HClO₃+Cl₂+H₂O

Kinetic studies have indicated that both decomposition pathways are pH dependant and increase with concentration, temperature, and exposure to light. Furthermore, the first process can be accelerated by catalysts, and the second process can be accelerated in the presence of other electrolytes, notably chloride ions. Due to decomposition, hypochlorite solutions can be more stable than hypochlorous acid solutions. For this reason, commercial solutions often have neutral or alkaline pH, which causes the free chlorine to exist as hypochlorite and not hypochlorous acid.

Although one does not intend to be bound to any particular theory, it is believed that the low chloride ion content is one of the main reasons for the unusual and advantageous stability over time of the electrolytic acid water, both to evaporation and self decomposition. Preferably, the amount of chlorides both at the beginning and at the end of the electrolytic process is low (200 ppm or lower), so that the water comprises chlorine in the form of HClO. For example, in a particular embodiment, the water can comprise ˜50 ppm of free chlorine, and lower than 200 ppm of chloride ions. At pH 2.80, this corresponds to about 99% HClO, and 1% dissolved gaseous chlorine.

The conductivity of the water preferably ranges from 900 to 1800 uS/cm, and more preferably ranges from 1000, 1100, 1200, or 1300 to 1400, 1500, 1600, or 1700 uS/cm. The free chlorine content of the water preferably ranges from 20 to 80 ppm, more preferably ranges from 30 or 40 to 60 or 70 ppm, and most preferably is about 50 ppm. The chloride ion content of the water preferably ranges from 150 to 250 ppm, more preferably ranges from 160, 170, 180 or 190 to 210, 220, 230, or 240 ppm, and most preferably is about 200 ppm. The chlorite content of the water preferably ranges from 50 to 150 ppb, more preferably ranges from 60, 70, 80 or 90 to 110, 120, 130, or 140 ppb, and most preferably is about 100 ppb. The chlorate content of the water preferably ranges from 0.5 to 1.5 ppm, more preferably ranges from 0.6, 0.7, 0.8, or 0.9 to 1.1, 1.2, 1.3, or 1.4 ppb, and most preferably is about 1 ppm.

Due to its chemical composition and acidity, the free chlorine in the water can be present in the form of hypochlorous acid (HOCl) and chlorine gas (Cl₂). The relative amount of HOCl and Cl₂ in the water preferably ranges from 99.9% HOCl and 0.1% Cl₂ to 95% HOCl to 5% Cl₂, more preferably ranges from 99.5% HOCl and 0.5% Cl₂ to 98.5% HOCl to 1.5% Cl₂, and most preferably is about 99.3% HOCl and 0.7% Cl₂.

Because the free chlorine in the water is present in the form of HOCl and Cl₂ in the ranges described above, the water can be highly stable against both evaporation and self decomposition. In an exposed, non-agitated state at a temperature of 25° C., the water preferably maintains a level of chlorine for a time of 2, 3, 4, 5, 6, 7, 8, 9, or 10 days. In an exposed, agitated state at a temperature of 25° C., the water preferably maintains a level of chlorine for a time of 8, 12, 16, 20, or 24 hours. In a closed state at a temperature of 25° C., the water preferably maintains about 90% of the free chlorine after a time of 3 months, and about 85% of the free chlorine after a time of 12 months. In a closed state at a temperature of 30° C., the water preferably maintains about 90% of the free chlorine after a time of 3 months, and about 80% of the free chlorine after a time of 12 months. In a closed state at a temperature of 40° C., the water preferably maintains about 90% of the free chlorine after a time of 3 months, and about 85% of the free chlorine after a time of 12 months.

Making Electrolytic Acid Water

The electrolytic acid water can be prepared, for example, by using the methods and electrolysis devices described in PCT Publications WO 2008/131936 and WO 2007/048772. The contents of said applications are hereby incorporated by reference as if fully set forth herein.

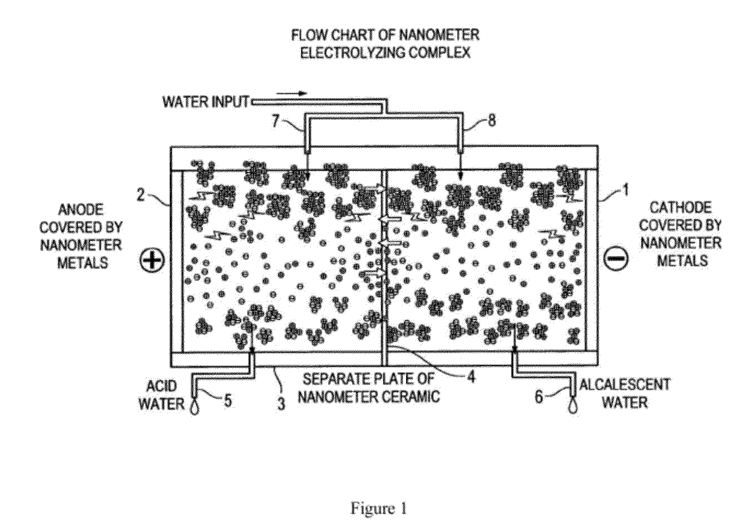

Referring now to FIG. 1, the electrolysis device can comprise an electrolysis chamber 3 divided into two portions by a membrane 4, and a pair of electrodes 1 and 2 within said chamber.

Preferably, both electrodes of the device are nano-coated electrodes as defined below. However, the advantages in terms of low cost and efficiency of the electrolysis process, as well as the advantages in terms of water stability over time, can be obtained also if only one of the two electrodes is nano-coated as defined above.

Preferably, the device according to the invention also comprises a membrane 4 adapted to divide the at least one chamber into two half-chambers, wherein the half-chamber that contains the anode is termed an anode half-chamber, and the half-chamber that contains the cathode is termed a cathode half-chamber. The membrane is advantageously an ultrafiltration membrane which can occupy the chamber partially or totally.

The membrane 4 can be of the type used in conventional electrolytic cells, but is preferably based on size exclusion technology at the nano-scale. Preferably, the membrane is made of ceramic material with open porosity, coated with metallic nano-particles, preferably nano-particles of oxides of zirconium, yttrium, aluminum or mixtures thereof. The metallic nano-particles used to make the coating are preferably in powder form. As regards the size distribution within the powder, preferably an amount at least equal to 70%, 75%, or 80% by weight of the particles that are present in the powder, more preferably at least equal to 85%, have a particle diameter ranging from 30 to 100 nm, 40 to 70 nm, or 50 to 60 nm.

By resorting to nanometer particles to manufacture the membrane 4, the average pore size of the final membrane has been found to be extremely constant over time and adaptable according to the requirements of how the water is to be processed. Preferably, the average pore size is from about 120 to about 180 nm (mean or median). Size constancy over time and constancy of the pore dimensions themselves are two aspects which differentiate the ceramic membrane described here from the textile membranes conventionally used in equivalent devices (which are instead subject to rapid deterioration over time). It is preferred that at least 50%, 70%, 90%, 95%, 98% or 99% of the pores have a diameter between 120 and 180 nm. These aspects have shown a positive effect on the stability of the water obtained after electrolysis, where this effect combines with, and augments, the stabilizing effect produced by the use of an electrode as defined above.

Importantly, the nano-sized dimensional features of the membrane and electrodes enhance the amount of active surface per unit of geometric surface, which creates a high apparent current density (i.e. the current intensity per unit of geometric surface). As a result, a high current intensity (ampere) and electric potential (voltage) can be provided to the solution, which can impart unique chemical and biological characteristics to the water. Preferably, the water is produced by applying a current intensity in the range of about 100 to about 39 ampere (24 to 18 volt) to a diluted sodium chloride solution in deionized water. By applying the high current intensity, a chemical composition of a low chloride ion content, low chlorite and chlorate content, and high hypochlorous acid content can be achieved.

The amount of current applied to the water preferably ranges from 30 to 120 ampere, more preferably ranges from 40, 50 or 60 ampere to 90, 100, or 110 ampere, and most preferably is about 80 ampere. The amount of voltage applied to the water preferably ranges from 15 to 35 volt, more preferably ranges from 16, 17, or 18 volt to 22, 23, or 24 volt, and most preferably is about 20 volt.

In a preferred electrolysis device, each half-chamber is connected to the outside of the device through:
openings 7 and 8 arranged in the upper part of the half-chamber from which the water to be subjected to electrolysis is inserted, and
additional openings 5 and 6 arranged in the lower part of the half-chamber which can act as a discharge for the resulting acid and alkaline fractions (referenced as “acid water” and “alkalescent water” in FIG. 1). The second opening on the lower part of each half chamber is provided with closure means (not shown) which is adapted to prevent the water that has not yet separated from leaving the half-chamber and are adapted to be opened at the end of the electrolytic process.

With specific reference to FIG. 1, the operating mechanism of a device as described above provided with all the essential and optional elements that have been listed, therefore entails treating water by introducing it from above, by means of the water input ducts, into the two half-chambers of the main chamber. Here, the water, under the action of the cathode and of the anode previously connected to the negative and positive poles of an electric voltage source, is split into positive and negative ions, which, as is known, are attracted by the respective opposite poles. In passing from one half-chamber to the other, the nano-porous membrane acts as a filter for said ions and for any charged particles, allowing only the particles of sufficiently small size to pass.

The water input to the unit can be characterized by its conductivity, preferably measured in μS/cm. Thus, for example, the water can be described by the consistency of conductivity in the water input. For example, the conductivity should vary by no more than 50, 20, 10, 5 or even 2 μS/cm, or 100, 50, 20 or 10%. The water may also be described by the conductivity of the water itself. The conductivity can range from 0.5, 1.0 or 1.5 μS/cm to 50, 25, 10, 5 or even 3 μS/cm, based on any selection of endpoints. The conductivity preferably ranges from 0.5 to 10 or 0.5 to 3 μS/cm, and the most preferred conductivity is about 2 μS/cm. It has been discovered that by controlling the consistency of the conductivity, and by lowering the conductivity to the preferred values, one is able to obtain much more consistent quality electrolyzed water, with a consequent reduction in NMR half line width. Suitable types of water for input into the unit include reverse osmosis water, deionized water, and distilled water. A preferred type of water due to its constant conductivity is osmotic water prepared by reverse osmosis.

The water preferably contains sodium chloride, or some other alkali metal salt, to facilitate the electrolysis. The sodium chloride is preferably pharmaceutical grade. The quantity of sodium chloride contained in the water is such that the water obtains a specific level of conductivity. The conductivity of the input solution preferably ranges from 50 μS/cm to 100 μS/cm, more preferably ranges from 150 μS/cm to 200 μS/cm, and most preferably is about 200 μS/cm.

Also of importance, the filter prevents the transmission of heavy metals from one chamber to the other. Thus, by introducing the water into the acidic or alkaline chamber, one is able to produce alkaline or acid water having practically no contamination by metallic radicals (or at least beyond the limits of detection).

A method of using such a unit for making electrolytic acid water having a NMR half line width using ¹⁷O-NMR of from about 45 to less than 51 Hz comprises:

(a) providing an electrolysis unit comprising: (i) a cathode chamber, an anode chamber, and a filter separating said chambers (preferably characterized by a porosity that allows ionized fractions of nano-clustered H₂O to pass, such as when the porosity is predominantly characterized by pores of from about 120 to about 180 nm in diameter (preferably having a mean diameter between 120 and 180 nm)); and (ii) a cathode situated in said cathode chamber and an anode situated within said anode chamber, wherein at least one of said anode and cathode is coated by a residue of particles in which greater than 70% by weight of said particles have a diameter of from 40 to 100 nm;

(b) introducing a solution of water and an alkali metal into one or both of said chambers; and

(c) applying an electric potential to said anode and said cathode, for a time and to an extent sufficient to produce electrolyzed acidic water having a NMR half line width using ¹⁷O of from about 45 to less than 51 Hz.

### EXAMPLES

Examples, as shown below, have demonstrated that the acidic nanoclustered water has anti-infectious, tissue healing, repair and regeneration, and other medical uses described above. The examples have also shown that that acidic electrolyzed water can remain stable for significantly longer period of time than previous electrolyzed oxidizing waters. It is noted that these examples merely serve the purpose of illustrating the present invention, and should not be construed as limiting the scope of the invention.

### Example 1

Objective

Determine the efficacy of acidic nanoclustered water (ANW) against several strains of bacteria, viruses and fungi.

Bacterial Activity

Bacterial activity was assessed with the method of UNI (Italian Organization for Standardization) EN 1040 (quantitative suspension test for the evaluation of basic bactericidal activity of chemical disinfectants and antiseptics). According to this method, a substance is classified as bactericidal for a specific microorganism if it reduces the bacterial count by at least 5-log₁₀ following 5 minutes of contact at 20° C. ANW solutions at three different concentrations (80%, 50%, and 25%) were tested against two strains of bacteria known to cause eye infections, Staphylococcus aureus (ATCC 6538) and Pseudomonas aeruginosa (ATCC 15442). Table 3 below shows the antibacterial effect of the three different concentrations of ANW, with viability reduction values expressed as the log₁₀ reduction.

TABLE 3
Antibacterial Effect of ANW

| | | Viability Reduction | | |
|---|---|---|---|---|
| Species | Solution | 80% | 50% | 25% |
| Staphylococcus aureus | ANW | >5.41 | >5.41 | 5.35 |
| (ATCC 6538) | | | | |
| Pseudomonas aeruginosa | ANW | >5.48 | <4.10 | <4.10 |
| (ATCC 15442) | | | | |

As shown in Table 3, ANW can be classified to be a bactericidal against both strains at a concentration of 80%.

Bacterial activity was also assessed against the same two strains of bacteria in the presence of 5% of human blood in the medium as organic soil interference. Viability reduction was assessed after 10, 30, 60 and 120 minutes of exposure to pure ANW at 31° C. Table 4 below shows the antibacterial effect of the pure ANW at each time point, with viability reduction values expressed as the log₁₀ reduction.

TABLE 4
Antibacterial Effect of ANW in Presence of 5% Human Blood

| | | Viability Reduction | | | |
|---|---|---|---|---|---|
| Species | Solution | 10 min | 30 min | 60 min | 120 min |
| Staphylococcus aureus | ANW | >5.6 | >5.6 | >5.6 | >5.6 |
| (ATCC 6538) | | | | | |
| Pseudomonas aeruginosa | ANW | >5.6 | >5.6 | >5.6 | >5.6 |
| (ATCC 15442) | | | | | |

As shown in Table 4, pure ANW was demonstrated to have a bactericidal effect against both strains at the lowest tested time point of 10 minutes.

Bacterial activity was also assessed against Propionibacterium acnes bacteria in the presence of 1% fetal bovine serum in the medium as organic soil interference. Viability reduction was assessed after 1, 5, 15 and 30 minutes of exposure to pure ANW at 31° C. Table 5 below shows the antibacterial effect of the pure ANW at each time point, with viability reduction values expressed as the log₁₀ reduction.

TABLE 5
Antibacterial Effect of ANW in Presence
of 1% Fetal Bovine Serum

| | | Viability Reduction | | | |
|---|---|---|---|---|---|
| Species | Solution | 1 min | 5 min | 15 min | 30 min |
| Propionibacterium acnes | ANW | >6.9 | >6.9 | 5.3 | >6.9 |
| (ATCC 11827) | | | | | |

As shown in Table 5, pure ANW was demonstrated to have a bactericidal effect at the lowest tested time point of 1 minute.

For testing the efficacy of ANW versus Methicillin Resistant Staphylococcus Aureus (MRSA), agent responsible of impetigo, an advanced testing substrate, that effectively mimics the surface properties of the human skin, was used. Following inoculation of the microorganism, the demarcated area of carriers (1″×1″ inch) were sprayed with 1.5 ml of ANW or were treated with a spread pea size amount of mupirocine ointment, the standard care treatment. After 30 minutes of exposure, each carrier was neutralized and assayed for survivors. ANW was proved to exert a strong antibacterial effect: at 30 minutes of exposure it was able to reduce of 99.99% (4.68 Log 10) the survival of MRSA, vs the 60.1% (0.40 Log 10) obtained by the marketed formulation of mupirocin.

Cornea Infection

The purpose of this study was to evaluate the ocular efficacy of ANW after multiple instillations in rabbit which underwent a corneal bacterial injection. Six animals was selected and subjected to bilateral cornea infection with Staphylococcus aureus (ATCC 25923 strain). Starting from the day of the infection, the animals were subjected to 4 washes daily/eye. Each treatment was performed with 15 ml (×15 sec.) of ANW for each eye of 3 animals and 15 ml (×15 sec.) of saline solution for each eye of other 3 animals. After 4 days of treatment the animals were sacrificed for the eyes sampling. ANW-treated animals showed a significantly lower number of viable bacteria compared to that observed in animals treated with saline solution as reported in Table 6 below.

TABLE 6
Ocular Efficacy of ANW after multiple instillations in rabbit

| | |
|---|---|
| | Counting of |
| | Staphylococcus aureus |
| Animal | ufc/cornea |

| | n. | right eye | left eye |
|---|---|---|---|
| Group 1 | 4 | 30 | <10* |
| ANW treated | 5 | 70 | 40 |
| | 6 | <10* | <10* |
| | mean | 37 | 20 |
| Group 2 | 7 | 1850 | 1200 |
| Saline solution | 8 | 4300 | 4550 |
| treated | 9 | 3000 | 2800 |
| | mean | 3050 | 2850 |

*= values <10 were considered equal to 10

Fungal Activity

Fungal activity was assessed with the method of UNI EN 1275 (quantitative suspension test for the evaluation of basic fungicidal activity of chemical disinfectants and antiseptics). According to this method, a substance is classified as fungicidal for a specific microorganism if it reduces the fungi count by at least 4-log₁₀ following 15 minutes of contact at 20° C. ANW solutions at three different concentrations (80%, 50%, and 25%) were tested against two strains of fungus known to cause infections, Candida albicans (ATCC 10231) and Aspergillus niger (ATCC 16404). Table 7 below shows the antifungal effect of the three different concentrations of ANW, with viability reduction values expressed as the log₁₀ reduction.

TABLE 7
Antifungal Effect of ANW

| | | Viability Reduction | | |
|---|---|---|---|---|
| Species | Solution | 80% | 50% | 25% |
| Candida albicans | ANW | >4.37 | <3.09 | <3.09 |
| (ATCC 10231) | | | | |
| Aspergillus niger | ANW | <3.12 | <3.12 | <3.12 |
| (ATCC 16404) | | | | |

As shown in Table 7, ANW can be classified to be a bactericidal against Candida albicans (ATCC 10231) at a concentration of 80%.

ANW was also able to provoke a >99.99% reduction of Trichophyton Mentagrophytes survivors in vitro after 5 minutes exposure time [Time Kill protocol (ASTM, E2315-03)], of Trichophyton Rubrum on a skin contaminated carrier after 10 minutes exposure time.

Viral Activity

Viral activity was assessed against Human Immunodeficiency Virus type 1 (HIV-1), Herpes Simplex Virus type 1 (HSV-1), and Herpes Simplex Virus type 2 (HSV-2) in the presence of 5% fetal bovine serum in the medium as organic soil interference. For HIV-1, viability reduction was assessed after 10 minutes of exposure to pure ANW at 21.5° C. For HSV-1 and HSV-2, viability reduction was assessed after 5 minutes of exposure to pure ANW at 35° C. Table 8 below shows the antiviral effect of the pure ANW on each virus, with viability reduction values expressed as the log₁₀ reduction.

TABLE 8
Antiviral Effect of ANW

| | | | Viability |
|---|---|---|---|
| Species | Solution | Exposure | Reduction |
| HIV-1 (strain HTLV-III_{b}) | ANW | 10 min at 21.5° C. | >4.5 |
| HSV-1 (ATCC VR733) | ANW | 5 min at 35° C. | <5.5 |
| HSV-2 (ATCC VR734) | ANW | 5 min at 35° C. | 4.25 |

As shown in Table 8, ANW can inactivate the tested viruses.

### Example 2

Objective

Determine the efficacy of acidic nanoclustered water (ANW) at promoting corneal healing, skin wound healing and cataract healing.

Corneal Ulceration Healing

Corneal healing activity was assessed in an in vivo rabbit model. Corneal eye wounds were experimentally provoked in 8 rabbits. The left and right eyes were then treated with ANW and saline, respectively, by applying 100 μl of the solutions 4 times per day for 14 consecutive days. On the 4^{th}, 9^{th}, and 14^{th} day after the surgery, images of each wound were taken under a slit lamp microscope and the area of each wound was calculated with the software Topcon IMAGENET 2000. The wound area was then used to calculate the wound healing rate (WHR) using the following equation:

WHR=100(1−(wound area at timing point)/(initial wound area))

Table 9 below shows the Wound Healing Rate of ANW and saline treated eyes at 4, 9, and 14 days.

TABLE 9
Corneal Ulceration Healing Effect of ANW and Saline

| | Wound Healing Rate (WHR) | | |
|---|---|---|---|
| | Day 4 | Day 9 | Day 14 |
| ANW | 77.78 ± 9.06 | 83.32 ± 12.23 | 87.20 ± 13.16 |
| Saline | 71.84 ± 19.38 | 63.45 ± 23.02 | 64.23 ± 28.28 |
| T-value | 1.36 | 3.73 | 3.61 |
| P-value | >0.05 | <0.05 | <0.05 |

As shown in Table 9, ANW was significantly more effective than saline in corneal ulcer healing at the latter two of the three time points.

The wounds were also observed daily for the presence of wound closure. On day 14, it was observed that half of the corneal ulcers treated with ANW were healed, while some corneas treated with saline were still presenting a large ulcer. Exemplary photographs (not shown) were taken of the corneal wounds in 3 of the rabbits on day 14.

The wounds were also observed daily for the presence of infections and inflammation. ANW was observed to reduce inflammation after injury. Furthermore, two of the eyes treated with saline were seriously infected with hypopyon, and the inflammation of these corneas was too intensive to identify the pupil. Exemplary photographs and histological images (not shown) were obtained of the inflammation in 2 of the rabbits.

Histological evaluation also was used to observe regeneration of the cornea and scarring. ANW was observed to increase regeneration and reduce scarring as compared to saline. Furthermore, epithelium deficiency was observed in all of the eyes treated with saline, and none of the eyes treated with ANW. Histological images (not shown) depicting scarring of cornea wounds were taken in 3 of the rabbits.

The wounds were also observed for the presence of angiogenesis. Neovascularization was observed in 35% of the corneas treated with saline, and none of the eyes treated with ANW. Exemplary photographs (not shown) depicting angiogenesis were taken of 2 of the rabbits.

Cataract Healing

Cataract healing activity was assessed in an in vivo rat model. Cataract was induced by intraperitoneal injection of d-galactose in 1 rat at a dose of 10 g/kg per day (twice/day). The left and right eyes were then treated with ANW and saline, respectively, by applying 1 drop of the solutions 4 times per day for 30 consecutive days. On day 30, it was observed that the cataract treated with ANW was significantly healed as compared to the cataract treated with saline. Photographs (not shown) depicting the cataracts were taken on day 30.

In Vitro Full-Thickness Skin

On a model of a full-thickness skin injured in vitro, the gene expression of several markers of the wound healing process were analysed. The application of ANW on the injured tissue led to a significant increase of the expressions of Collagen I, Collagen VII and MMP-9 (a metalloprotease) as the early markers involved in tissue repair process. No effects were shown on the expressions of Fibronectin, TNF-α and Integrin β-1. The histo-morpholological study showed a very good recovery of the tissue after injury treated with ANW

Wound Healing

The objective of the study was the evaluation of the effects on “chronic” wound healing of ANW vs commercial competitor (more or less same quantity of Free Chorine but PH around 7: that means a different species of Free Chlorine in fact the commercial competitor contains more or less 30 ppm of HClO and 20 ppm of ClO—) treatment in terms of both time to wound closure, or wound healing rate, and scar tissue histological organization. The animal model utilized for this study was the rabbit ischemic ear model. On each of the 12 rabbits used for the experiment, on one ear (left) ischemia was surgically induced while the other was left normal. On each ear, 4 full thickness wounds were created with a trephine having a drilling tip of 6 mm diameter. Wounds were daily observed to check the state of the wounds in term of presence infections, exudates and wound closure. At days 3^{rd}, 7^{th}, 11^{th}, 15^{th} and 19^{th}, wounds area were measured under slit lamp microscope. Histological evaluation was made on two animals (rabbit n. 6 and n. 8) on day 14^{th} and in all the rest at day 19^{th}. After surgery, ear temperature difference between ischemic and not ischemic ear, was on average of 5° C. This difference was maintained for the first 7 days. After then it started to decrease because of the removal of the artery closure, reaching, at the end of the study, 1° C. In ischemic ear, at day 15^{th} only 2 wounds were healed and both in the ANW treated wounds. The total number of closed wounds observed in ischemic ear at the end of the study were 10/14, 5/11 and 1/14 for ANW, Commercial competitor and Saline treatments, respectively [see Table 10].

TABLE 10
Number of healed wounds in the ischemic
ear group at day 15th and 19th.

| | | |
|---|---|---|
| | Day 15^{th} | Day 19^{th} |
| TREAT- | Healing | Healing |

| MENTS | Yes | No | Yes | No |
|---|---|---|---|---|
| ANW (n = 14) | 2 | 12 | 10 | 4 |
| Comm. | 0 | 11 | 5 | 6 |
| competitor | | | | |
| (n = 11) | | | | |
| Saline (n = 14) | 0 | 14 | 1 | 13 |

In non-ischemic ear, already at day 15^{th} all the wounds treated with ANW were healed while in Commercial competitor treated wounds the healed wounds were 8/12 and for Saline treated group the healed wounds were 8/14. At the end of the study 100% of wounds area were closed in ANW (n=14) and Commercial competitor (n=12) treated wounds whilst in saline treated wounds two wounds were not completely healed [see Table 11].

TABLE 11
Number of healed wounds in the non ischemic
ear group at day 11th, 15th and 19th.

| | Day 11^{th} | | Day 15^{th} | | Day 19^{th} | |
|---|---|---|---|---|---|---|
| TREAT- | | Not | | Not | | Not |
| MENTS | Healed | Healed | Healed | Healed | Healed | Healed |
| ANW (n = 14) | 10 | 4 | 14 | 0 | 14 | 0 |
| Comm | 3 | 9 | 8 | 4 | 12 | 0 |
| competitor | | | | | | |
| (n = 12) | | | | | | |
| Saline | 4 | 10 | 8 | 6 | 12 | 2 |
| (n = 14) | | | | | | |

No infections were detected in ANW treated wounds in both ischemic and non-ischemic conditions while infections were recorded in Commercial competitor and Saline treated wounds in both ischemic and non-ischemic conditions [see Table 12].

TABLE 12
Number of infected wounds and total time of infection

| | |
|---|---|
| ISCHEMIC | NON-ISCHEMIC |

| | Total | Total | Total | Total |
|---|---|---|---|---|
| TREAT- | infected | days of | infected | days of |
| MENTS | wounds | infection | wounds | infection |
| ANW | 0/14 | 0 | 0/14 | 0 |
| Comm | 3/11 | 15 | 1/12 | 2 |
| competitor | | | | |
| SALINE | 5/14 | 24 | 1/14 | 8 |

NB: The total days of observation are: 266 for ANW, 209 for Commercial competitor; 266 for Saline in ischemic group and 266 for ANW, 228 for Commercial competitor; 266 for Saline in non-ischemic group

Exudates, although at different extent, was recorded in all groups in both ischemic and non-ischemic conditions [see Table 13].

TABLE 13
Number of wounds with exudates and total time of persistence

| | ISCHEMIC | | | NON-ISCHEMIC | | |
|---|---|---|---|---|---|---|
| | Total | Total | Wound | Total | Total | Wound |
| | wounds | number of | with | wounds | number of | with |
| | with | days with | abundant | with | days with | abundant |
| TREATMENTS | exudates | exudates | exudate | exudates | exudates | exudate |
| ANW | 4/14 | 14 | 1/14 | 4/14 | 5 | 0/14 |
| Comm. Competitor | 6/11 | 19 | 1/11 | 6/12 | 19 | 0/12 |
| SALINE | 7/14 | 37 | 2/14 | 3/14 | 23 | 1/14 |

NB: The total days of observation are: 266 for ANW, 209 for Commercial competitor; 266 for Saline in ischemic group and 266 for ANW, 228 for Commercial competitor; 266 for Saline in non-ischemic group

Histological evaluation revealed a more neat granulation tissue and extracellular matrix deposition in ANW treated wounds respect to Commercial competitor or Saline treated wounds. Results herein obtained indicate that in both ischemic and non-ischemic wounds ANW performed better than Commercial competitor which performed better than Saline in terms of both wound healing and antibacterial activity.

### Example 3

Objective

Determine the safety of acidic nanoclustered water (ANW) in systemic and topical applications.

In Vitro Studies

Citotoxicity was assessed with the method of ISO (International Organization for Standardization) 10993-5. According to this method, a substance is classified based on its effect on a cell culture. 100 μl of pure ANW was applied to a cell culture of murine fibroblasts L-929 and the cells were evaluated after 24 hours of incubation at 37° C. Some malformed cells were observed after the period of incubation. Based on these results, ANW was defined as “slightly cytotoxic” (grade 1 of 4).

Mutagenicity was assessed with the method of OECD 471. According to this method, a substance is classified based on its ability to induce point mutations in bacteria. Five mutant strains of Salmonella typhimurium (TA 1535, TA 1537, TA 98, TA 100, and TA 102) were studied both in the presence and in the absence of ANW. Based on the results of a reverse mutation assay (Ames' test), the substance ANW was defined as non mutagenic.

Systemic Toxicity Studies

Acute toxicity was assessed with the method of ISO 10993-11, 2006, Biological Evaluation of Medical Devices—Part 11: Tests for Systemic Toxicity. According to this method, a substance is classified not toxic if animals injected with the substance do not show a significantly greater biological reaction than animals treated with a control article. 10 female Swiss albino mice were injected by intraperitoneal route with either ANW or saline in the amount of 50 mL/kg. The animals were observed for clinical signs immediately after injection, and at 4, 24, 48, and 72±2 hours after injection. ANW did not induce a significantly greater biological reaction than the control, and was therefore classified as not toxic.

Skin Irritation Studies

Dermal irritation following acute exposures was assessed with the method of ISO 10993-10. 0.5 mL of pure ANW was applied with a patch on the shaved skin of three male albino rabbits. The patch was held on the skin by means of a non-irritating adhesive plaster for 4 hours. The skin reaction was observed upon removal of the patch and 24, 48, and 72 hours after removal. No sign of either erythema or edema was observed. Based on these results, ANW was determined to be non-irritating for skin, which a Skin Irritation Index of 0.00.

Dermal irritation following repeated exposure also was assessed with the method of ISO 10993-10. Three male New Zealand rabbits were treated 5 days a week for 4 weeks with two consecutive daily administrations of 5 mL of pure ANW or saline as a control applied with a patch for one hour. The skin reaction was before and after each application throughout the entire 4 week period. No sign of either erythema or edema was observed. Furthermore, upon sacrifice, no signs of inflammation were detected in histological images. Based on these results, ANW was determined to not exhibit any significant irritancy in the skin.

Skin Sensitization Studies

Delayed-type skin hypersensitivity was assessed with the method of ISO 10993-10:
Guinea-Pig Maximization test. The test used 15 albino female Hartley guinea pigs (10 treated and 5 control). The injection phase (Day 0) was carried out by administering three 0.1 mL intradermal injections to each animal: (a) complete Freund's adjuvant, (b) either pure ANW (test) or saline (control), (c) either ANW (test) or saline (control) mixed together with complete Freund's adjuvant. A skin massage with 1 mL SLS 10% was then performed on Day 6. The induction phase (Day 7) was carried out by applying 1 mL of either the test or the control, left in place for 48 hours with an occlusive patch. The challenge was carried out on Day 21 through the application to each animal (both treated and control) of dressings with of 1 mL of ANW on the right side and 1 mL of saline on the left side, left in place for 24 hours.

Assessments were carried out on the 23rd day (24 hours after patch and removal) and on the 24th day (48 hours after patch and removal). The intensity of erythem and/or edema was evaluated according to the Magnusson and Kligman scale from 0 to 3.

No abnormalities were observed in either the treated or the control animals. Based on these results, ANW was determined to not exhibit delayed contact dermatitis potential.

Ocular Irritation Studies

Ocular irritation was assessed with the method of ISO 10993-10. In a first experiment, three New Zealand white rabbits were treated by instilling 0.1 mL of pure ANW into the left eye, leaving the right eye untreated as a control. The eyes were examined 1, 24, 48 and 72 hours after instillation through fluorescein staining and slit-lamp observation. No signs of irritation were observed in any of the eyes. Based on these results, ANW was determined to be a non-irritant for the ocular tissue of New Zealand White rabbits.

In a second experiment, ocular irritation was again assessed with the method of ISO 10993-10. Three New Zealand white rabbits were treated by instilling 0.1 mL of pure ANW into the left eye as a test, and instilling 0.1 mL of NaCl containing water (saline) into the right eye as a control. The treatment was repeated for 30 consecutive days. No signs of irritation were observed in any of the test or control eyes at any of the observation points. Based on these results, the test article ANW was determined to be a non-irritant for the ocular tissue of New Zealand White rabbits.

Oral Irritation

The primary buccal irritation test of ANW was carried out following the protocol ISO 10993-10. Six female Golden Syrian hamsters were given 0.5 mL of ANW, placed into one cheek pouch of each animal. The other cheek pouch was untreated. Pouch mucosa was exposed to ANW for a minimum of 5 min, repeated hourly for 4 hours. The animals were observed for 24 hours for signs of local intolerance, then sacrificed and histology of the oral mucosa was performed. No signs of irritation were observed, therefore the test article ANW can be considered non-irritant for the buccal tissues of the hamster.

Primary Vaginal Irritation

The potential of ANW to produce vaginal irritation after five consecutive days of application was studied in 3 female New Zealand White rabbits (and 3 untreated rabbits as controls) following the protocol ISO 10993-10. A volume of 1.0 mL of ANW was inserted in vagina of 3 animals, daily for 5 consecutive days. Animals were observed daily and until 24 hours after the last application for signs of irritation, erythema, edema. At the end of the study period, the rabbits were sacrificed and morphology and histology of the vaginal tissue performed. No signs of irritation were observed, therefore the test article ANW can be considered non-irritant for the vaginal tissue of New Zealand White rabbits.

Keratolytic Effect

The prediction of the exfoliating/keratolytic potential of ANW was made through cytotoxicity testing (MTT assay) on in vitro reconstituted human 3D skin with a corneum layer, for comparison with a well-known keratolityc agent (10% glycolic acid) and with a moderate irritating cytotoxic agent (1% SLS). The assay was carried out on a three-dimensional reconstituted human skin model, composed of human epidermal keratinocytes that have been proliferating and differentiating in vitro in peculiar conditions in order to build up a well-differentiated stratum corneum. The objective of these assays were to assess quantitatively the effects of the test materials and controls on skin cell survival (MTT assay), that is able to evaluate the damages to the germinative, and hence viable, layer of the epidermis. When the damage of the corneum layer is stronger, as it happens with keratolytic agents, the cytotoxic effects of the germinative layer are faster to develop. While a moderate irritant, as 1% SLS, does not cause any cell death after 15′ and only about 30% of cell death after at least 2 h treatment, a moderate keratolytic agent such as 10% Glycolic acid is able to cause a 50% death after 15′ and a 90% cell death after 2 h, with a more direct and stronger effect due to the keratin lysis and to the consequent brake through the corneum layer. Thirty μL of each substance have been applied to the cell culture and incubation at 37° C. was performed for 15 and 120 minutes respectively. ANW did not show any keratolytic effect, in that it did not cause any significant cell death in the germinative layer. A well-known exfoliating agent, such as 10% glycolic acid produced 45.86% of cell death at 15′, while the irritating agent SLS showed its partial cytotoxic effect only after longer times exposure (27.69% after 2 hours).

Intramuscular Implantation Test

The purpose of the study was to evaluate ANW for local tissue responses and for the potential to induce local toxic effects after implantation in the muscle tissue of New Zealand White rabbits following the protocol ISO 10993-6. ANW was used to flush the implant sites with at least 10 mL of sample and sutured in situ into each of the paravertebral muscles of rabbits. The results indicate that the test article does not demonstrate any remarkable difference as compared to the control implant sites, when implanted for 2 weeks.

Hemolysis Test

The system for the determination of hemolytic activity of a test article when in contact with human blood was designed following the protocol ISO 10993-4. ANW was tested for its hemolytic capacity at neat (100%), 50% and 10% concentrations. 40.91% and 56.54% hemolysis was observed at neat and 50% concentrations. Only 3.03% hemolysis was observed when tested at 10% concentration.

Pyrogen Test

The purpose of the study was to determine the presence of chemical pyrogens in the material, in order to limit to an acceptable level the risks of febrile reaction following administration of the product to the patient. The study involved measuring the rise in temperature of New Zealand White rabbits following the intravenous injection of ANW in a dose not exceeding 10 mL per kg, within a period of not more than 10 minutes. Body temperatures were recorded at 0 hour and then at 30 minute intervals between 1 and 3 hours subsequent to injection. The test article is considered non-pyrogenic and meets the requirements of the pyrogen test, according to ISO 10993-11 guideline.

Phototoxicity

The in vitro 3T3 NRU phototoxicity test is based on a comparison of the cytotoxicity of a chemical when tested in the presence and in the absence of exposure to a non-cytotoxic dose of simulated solar light. Cytotoxicity in this test is expressed as a concentration-dependent reduction of the uptake of the vital dye Neutral Red when measured 24 hours after treatment with the test chemical and irradiation. Balb/c 3T3 cells are maintained in culture for 24 h for formation of monolayers. Two 96-well plates per test chemical are pre-incubated with eight different concentrations of the test substance for 1 h. Thereafter one of the two plates is exposed to the highest non-cytotoxic irradiation dose whereas the other plate is kept in the dark. In both plates the treatment medium is then replaced by culture medium and after another 24 h of incubation cell viability is determined by Neutral Red uptake. Cell viability is expressed as percentage of untreated solvent controls and is calculated for each test concentration. To predict the phototoxic potential, the concentration responses obtained in the presence and in the absence of irradiation are compared, usually at the IC50 level, i.e., the concentration reducing cell viability to 50% compared to the untreated controls. ANW did not show any phototoxic effect.

Summary of Toxicology Studies

Table 14 below shows a synopsis of the safety studies reported above in Example 3.

TABLE 14
Synopsis of ANW Safety Studies

| TITLE | STANDARD | RESULTS |
|---|---|---|
| CYTOTOXICITY | ISO 10993-5—GLP testing | SLIGHTLY CYTOTOXIC |
| | and reporting | |
| DELAYED-TYPE | ISO 10993-10—GLP testing | NON SENSITIZING |
| HYPERSENSIVITY | and reporting | |
| SALMONELLA | OECD 471-GLP testing and | NOT MUTAGENIC |
| TYPHIMURIUM | reporting | |
| REVERSE MUTATION | | |
| ASSAY | | |
| (tested concentration = | | |
| 50 mg/ml) | | |
| IN VITRO | OECD 432 | NOT PHOTOTOXIC |
| PHOTOTOXICITY | | |
| EVALUATION | | |
| ACCORDING TO OECD | | |
| 432 (3T3 NRU assay) | | |
| HEMOLYSIS | ISO 10993-4: 2002, as | 3.03% hemolysis when |
| | amended 2006—GLP testing | tested at 10% concentration |
| | and reporting | |
| PYROGENICITY | ISO 10993-11: 2006—GLP testing | NOT-PYROGENIC |
| | and reporting | |
| DIRECT SYSTEMIC | ISO 10993-11—GLP testing | NOT TOXIC |
| INJECTION TEST - ISO | and reporting | |
| PRIMARY OCULAR | ISO 10993-10—GLP testing | NOT IRRITANT |
| IRRITATION-ISO, | and reporting | |
| DIRECT CONTACT | | |
| REPEATED OCULAR | ISO 10993-10—GLP testing | NOT IRRITANT |
| IRRITATION: 30-DAY | and reporting | |
| OCULAR SAFETY | | |
| STUDY | | |
| ORAL IRRITATION | ISO 10993-10—GLP testing | NOT IRRITANT |
| TEST-ACUTE | and reporting | |
| EXPOSURE-ISO, | | |
| DIRECT CONTACT | | |
| PRIMARY VAGINAL | ISO 10993-10—GLP testing | NOT IRRITANT |
| IRRITATION-REPEAT | and reporting | |
| EXPOSURE-ISO | | |
| SKIN IRRITATION | ISO 10993-10—GLP testing | NOT IRRITANT |
| | and reporting | |
| SKIN IRRITATION | ISO 10993-10—GLP testing | NOT IRRITANT |
| Repeated exposure | and reporting | |
| (2 application/ | | |
| day for 4 weeks) | | |
| IN VITRO | bibliography | No keratolytic effect was |
| EVALUATION OF | | shown. |
| KERATOLYTIC | | |
| EFFECT OF A | | |
| PRODUCT ON | | |
| RECONSTITUTED | | |
| EPIDERMIS | | |
| INTRAMUSCOLAR | ISO 10993-6: 2007—GLP testing | No difference compared to |
| IMPLANTATION TEST | and reporting | the control (physiologic |
| | | solution) |

### Example 4

Objective

Determine the efficacy of acidic nanoclustered water (ANW) for modulating the activity of the immune system

In Vitro Study of PBMC Proliferation

The ability of ANW to inhibit the proliferation of peripheral blood mononuclear cells (PBMC) was assessed in an in vitro cellular model using 12 batches of PCMC. In the experiment, 4 of the batches were exposed to betamethasone (10 nM), a glucocorticoid steroid with anti-inflammatory and immunosuppressive properties, 4 of the batches were exposed to a 1:10 dilution of ANW, and the remaining 4 batches were exposed to a 1:20 dilution of ANW. Table 10 below shows the inhibition effect that was measured for each of the 12 batches.

TABLE 15
Inhibition of PBMC Proliferation

| | Batch 1 | Batch 2 | Batch 3 | Batch 4 | Mean |
|---|---|---|---|---|---|
| Betamethasone (10 nM) | 87.7% | 85.7% | 79.4% | 88.5% | 85.3% |
| Acidic Nanoclustered | 39.9% | 29.0% | 19.3% | 16.6% | 26.2% |
| Water (1:10) | | | | | |
| Acidic Nanoclustered | 17.3% | 22.0% | 4.0% | 7.0% | 12.6% |
| Water (1:20) | | | | | |

As shown in Table 15, ANW inhibited PBMC proliferation at both dilutions. These dilutions have previously been shown to not be significantly toxic on this cell type in vitro.

In Vitro Study of T-Cell Activation

The ability of ANW to modulate immunoregulatory cytokines was assessed in an in vitro cellular model using PBMC from 4 blood donors stimulated with purified protein derivatives from Mycobacterium tuberculosis (PPD), a prototypical Th1 antigen. In the experiment, batches of PBMC cells were stimulated with PPD alone, PPD plus betamethasone (10 nM), and PPD plus Acidic Nanoclustered Water (1:10 dilution). T-Cell activation was measured by testing levels of three cytokines: IL-10 (expressed in T-Cells), IFN-gamma (expressed in Th-1 cells), and IL-4 (expressed in Th-2 cells). FIG. 3 shows the levels of each cytokine as compared to non-stimulated PBMC.

As shown in FIG. 3, ANW up-regulated IL-10 production by stimulated PMBC in a statistically significant way (T test: p<0.05). Interleukin IL-10 is an important immunoregulatory cytokine. Its main biological function is to limit and terminate inflammatory responses, and to regulate the differentiation and proliferation of several immune cells. IL-10 deficiency is regarded as pathophysiologically relevant in inflammatory disorders characterized by a type 1 cytokine pattern such as psoriasis. Thus, the immune-modulating properties of ANW and, specifically, the IL-10 activating properties of ANW, suggest than ANW can be used as a direct therapeutic agent for several skin diseases.

### Example 5

Objective

Compare the stability of acidic nanoclustered water (ANW) with the reported stability of other electrolyzed waters of similar pH, ORP, and composition.

Comparison of ANW with Electrolyzed Oxidizing (EO) Water of Soo-Voo Len

The stability of ANW in open and closed conditions at 25° C. was compared with the stability of EO water as reported in the article “Effects of Storage Conditions and pH on Chlorine Loss in Electrolyzed Oxidizing (EO) Water”—Journal of Agricultural and Food Chemistry—2002, 50, 209-212 by Soo-Voon Len, et al.

In Soo-Voo Len, electrolyzed water with an acidic pH (2.5-2.6), OPR>1000 mV (1020-1120), and a free chlorine content ˜50 ppm (53-56 ppm) was generated with an ROX-20TA device manufactured by Hoshizaki Electric Inc. (Aichi, Japan) using a current intensity of 14 Ampere and 7.4 Volt. The article reports that in an open condition at 25° C., the chlorine in the electrolyzed water was completely lost after 30 hours when agitated, and after 100 hours when not agitated. Furthermore, as seen in FIG. 1 of the article, the free chlorine was almost completely lost after 10 hours in open, agitated, diffused light conditions. The article also reports that in a closed dark condition at 25° C., the free chlorine in the electrolyzed water decreased by approximately 40% after 1400 hours (about 2 months).

In comparison, ANW stored in an open condition at 25° C. without agitation completely lost chlorine after 240 hours (10 days), more than twice as long as the EO water in Soo-Voo Len (100 hours). Furthermore, ANW stored in an open condition at 25° C. with agitation and light completely lost chlorine after 24 hours, more than twice as long as the EO water in Soo-Voo Len (10 hours). Finally, ANW stored in a closed condition at 25° C. lost 8.44% of free chlorine after 3 months, less than a quarter as much as was lost from the EO water in Soo-Voo Len after about 2 months (40%).

Comparison of ANW with Electrolyzed Oxidizing (EO) Water of Shun-Yao Hsu

The stability of ANW in closed conditions at around 30° C. was compared with the stability of EO water as reported in the article “Effects of storage conditions on chemical and physical properties of electrolyzed oxidizing water”—Journal of Food Engineering 65 (2004) 465-471 by Shun-Yao Hsu, et al.

In Shun-Yao Hsu, the electrolyzed water of “formulation J” had an acidic pH (2.61), OPR=1147 mV, and a free chlorine content of 56 ppm. The article reports that in a closed condition at 25-30° C., the free chlorine in the electrolyzed water was 43 ppm after 21 days, a 23% loss. In comparison, ANW samples stored in closed conditions at 25° C., 30° C., and 40° C. without agitation lost 8.44%, 8.64%, and 15.43% of free chlorine after 3 months, 12.14% and 18.31% of free chlorine after 1 year at 25° C., 30° C. respectively and 19.13% of free chlorine after 6 month at 40° C.

### Example 6

The properties and composition of Acidic Nanoclustered Water were tested and found to have specifications as reported below in Table 16. The properties and composition Acidic Nanoclustered Water were also analyzed as reported below in Table 17. Preferably, the Acidic Nanoclustered Water for human application and industrial application has specifications as reported below in Table 18 and Table 19, respectively.

TABLE 16
Acid Water Specifications

| Test Item | Method | Specification |
|---|---|---|
| Appearance | Naked eye | Liquid |
| Odour | Smell | Characteristic |
| Colour | Naked eye | Colourless |
| pH | as is @ 25° C. | <3.00 |
| | by Mettler Toledo pHmeter | |
| | SevenMulti - | |
| | Potentiometric Determination | |
| | (Ph Eur. 2.2.3 - Current Ed.) | |
| OxidoReductive | as is @ 25° C. | >1100.0 |
| Potential | by Mettler Toledo combination | |
| ORP (mV) | redox electrode (P/N 51343200) | |
| | Potentiometric Tritation | |
| | (Ph Eur. 2.2.20 - Current Ed.) | |
| Conductivity | as is @ 25° C. | <1300 |
| (uS cm⁻¹) | | |
| Free Chlorine | Internal Method M37-07 | 40.0-70.0 |
| Assay (mg/l or | Spectrophotometric Method | |
| ppm) | source APAT IRSA CNR | |
| | HandBook Volume 2 - Ref 4080 | |
| Total Chlorine | Internal Method M37-07 | 40.0-70.0 |
| Assay (mg/l or | Spectrophotometric Method | |
| ppm) | source APAT IRSA CNR | |
| | HandBook Volume 2 - Ref 4080 | |
| Total Chlorine | Internal Method M37-07 | 40.0-70.0 |
| Assay (mg/l or | Iodometric Method | |
| ppm) | source APAT IRSA CNR | |
| | HandBook Volume 2 - Ref 4080 | |
| Chloride Assay | Internal Method M05-08 | <200.0 |
| ((mg/l or ppm) | Spectrophotometric Method | |
| | source APAT IRSA CNR | |
| | HandBook Volume 2 - Ref 4090 | |
| Chlorites | EPA 300.1, 1997 (as ClO₂) | <100 |
| (μg/l or ppb)) | | |
| Chlorates | EPA 300.1, 1997 | <10 |
| (mg/1 or ppm) | | |
| ¹⁷O-NMR (Hz) | ¹⁷O-NMR Spectrometer | <50 |
| Linewidth @ 50% | | |
| Heavy metals | ICP Method | <10 ppm |
| [Ag, As, Bi, Cd, | | |
| Cu, Hg, Mo, Pb, | | |
| Sb, Sn] | | |
| Yttrium | by EPA 200.8 1994 | <0.1 ppm |
| (ICP Method) | (0.1 μg/l detection limit) | |
| Zinc | by EPA 200.8 1994 | <0.1 ppm |
| (ICP Method) | (0.1 μg/l detection limit) | |
| Iridium | by EPA 200.8 1994 | <0.1 ppm |
| (ICP Method) | (0.1 μg/l detection limit) | |
| Titanium | by EPA 200.8 1994 | <0.1 ppm |
| (ICP Method) | (0.1 μg/l detection limit) | |
| Zirconium | by EPA 200.8 1994 | <0.1 ppm |
| (ICP Method) | (0.1 μg/l detection limit) | |
| Ruthenium | by EPA 200.8 1994 | <0.1 ppm |
| (ICP Method) | (0.1 μg/l detection limit) | |

TABLE 17
Acid Water Test Results

| | ANW | ANW | ANW |
|---|---|---|---|
| | LOT | LOT | LOT |
| | LCOVI/57 | LCOX/5 | LCOX/1 |
| Appearance | colourless | Same | Same |
| | liquid with light | | |
| | chlorine smell | | |
| | (like swimming | | |
| | pool) | | |
| Free Chlorine Assay (mg/l) | 53.1 | 48.6 | 49.9 |
| Spectrophotometric Method | | | |
| Total Chlorine Assay (mg/l) | 52.1 | 48.6 | 49.0 |
| Spectrophotometric Method | | | |
| Total Chlorine Assay (mg/l) | 60.6 | 54.9 | 56.7 |
| Iodometric Method | | | |
| Chloride Assay (mg/l) | 138 | 194.0 | 183.4 |
| UNI 24012 (Mercurimetric | | | |
| method) | | | |
| Chlorites μg/l (as ClO₂) by | <100 | 100 | <100 |
| EPA 300.1 1997 | | | |
| (detection limit 100 μg/l) | | | |
| Chlorates mg/l by EPA | 1.20 | 1.5 | 0.9 |
| 300.1 1997 | | | |
| (detection limit 0.1 mg/l) | | | |
| pH | 2.59 | 2.71 | 2.81 |
| (as is by Mettler Toledo pH | | | |
| meter Met Rohm 744) | | | |
| ORP by Mettler Toledo | 1151.8 | 1121.7 | 1110.5 |
| PT4805-60-88TE-S7/120 | | | |
| combination redo electrode | | | |
| ¹⁷O NMR (Linewidth @ | 45.76 | 45.33 | 46.07 |
| 50% - Hz) | | | |
| Heavy Metals | <10 ppm | <10 ppm | <10 ppm |
| (Ag, As, Bi, Cd, Cu, Hg, | | | |
| Mo, Pb, Sb, Sn) | | | |

TABLE 18
Human Acid Water Specifications

| Test Item | Method | Specification |
|---|---|---|
| pH | as is @ 25° C. | <3.00 |
| | by Mettler Toledo pHmeter | |
| | SevenMulti - Potentiometric | |
| | Determination | |
| | (Ph Eur. 2.2.3 - Current Ed.) | |
| OxidoReductive | as is @ 25° C. | >1000.0 |
| Potential | by Mettler Toledo combination | |
| ORP (mV) | redox electrode (P/N 51343200) | |
| | Potentiometric Tritation | |
| | (Ph Eur. 2.2.20 - Current Ed.) | |
| Free Chlorine | Internal Method M37-07 | 40.0-70.0 |
| Assay (mg/l or | Spectrophotometric Method | |
| ppm) | source APAT IRSA CNR | |
| | HandBook Volume 2 - Ref 4080 | |
| Percentage of | Internal Method M37-07 | >95% |
| HClO as a Free | Spectrophotometric Method | |
| Chlorine Species | source APAT IRSA CNR | |
| in Total Free | HandBook Volume 2 - Ref 4080 | |
| Chlorine (%) | | |
| Chloride Assay | Internal Method M05-08 | <200.0 |
| ((mg/l or ppm) | Spectrophotometric Method | |
| | source APAT IRSA CNR | |
| | HandBook Volume 2 - Ref 4090 | |
| 17O-NMR (Hz) | 17O-NMR Spectrometer | <50 |
| Linewidth @ 50% | | |

TABLE 19
Industrial Acid Water Specifications

| Test Item | Method | Specification |
|---|---|---|
| pH | as is @ 25° C. | <3.00 |
| | by Mettler Toledo pHmeter | |
| | SevenMulti - Potentiometric | |
| | Determination | |
| | (Ph Eur. 2.2.3 - Current Ed.) | |
| OxidoReductive | as is @ 25° C. | >1100.0 |
| Potential | by Mettler Toledo combination | |
| ORP (mV) | redox electrode (P/N 51343200) | |
| | Potentiometric Tritation | |
| | (Ph Eur. 2.2.20 - Current Ed.) | |
| Free Chlorine | Internal Method M37-07 | <500 |
| Assay (mg/l or | Spectrophotometric Method | |
| ppm) | source APAT IRSA CNR | |
| | HandBook Volume 2 - Ref 4080 | |

### Example 6

The stability of Acidic Nanoclustered Water compositions containing different amounts of chloride ion were tested both in storage and in the open air. The low chloride composition contained less than 200 ppm chloride, and the high chloride composition contained 1100 ppm chloride.

To test storage stability, the compositions were stored in a closed condition at 25° C. and 60% relative humidity, and were not agitated or exposed to diffused light. After 3 and 12 months, the low chloride composition had a loss of free chlorine of 8.44% and 12.14%, respectively. In contrast, the high chloride composition had a loss of free chlorine of 27.4% after only 3 months.

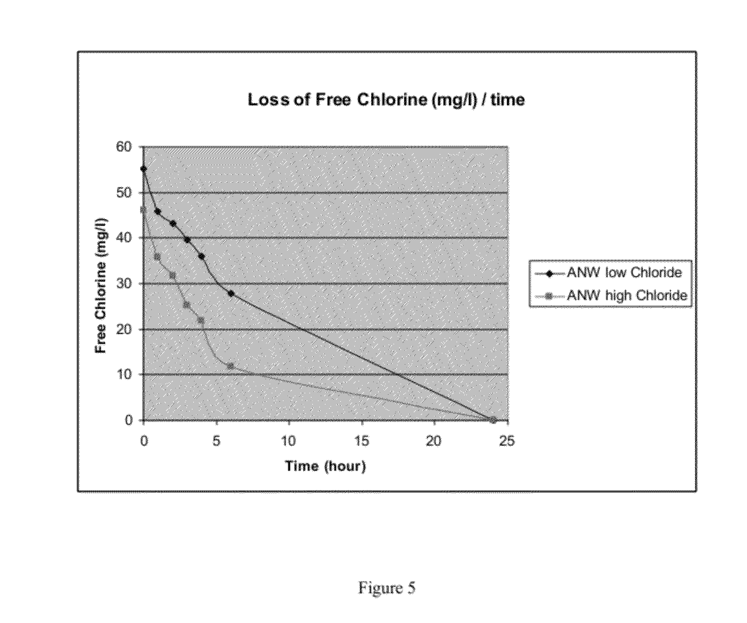

To test open air stability, the two compositions were kept open, agitated, and exposed to light for 24 hrs at a temperature of 30° C. As illustrated in FIG. 5, the high chloride composition lost free chlorine at a greater rate than the low chloride composition.

These results demonstrate that the stability of ANW is dependent of chlorine remaining HClO, which prevents both evaporation and decomposition.

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All references cited herein, including patents, patent applications, and published patent applications, are hereby incorporated by reference in their entireties, whether or not each is further individually incorporated by reference.

1) A method of treating or preventing an infection or infectious disease in a mammal, or sanitizing mammalian tissue, or treating or preventing psoriasis in a mammal, comprising topically administering to said mammal an electrolytic acid water comprising free chlorine, wherein:
a) greater than 95% of said free chlorine is present in the form of hypochlorous acid;
b) said water has a pH of from 0.5 to 5.0; and
c) said water has oxidation reduction potential (ORP) of greater than 1000 mV.

2) The method of claim 1, wherein said water is further characterized by a pH less than 3, free chlorine of from 40 to 70 ppm, and chloride of less than 200 ppm.

3) The method of claim 1, wherein said infection or infectious disease is bacterial, viral, fungal or parasitic.

4) The method of claim 1, wherein said infection or infectious disease is a bacterial infection selected from Anthrax, Bacterial meningitis, Botulism, Brucellosis, Campylobacteriosis, Cat scratch disease, Cholera, Epidemic Typhus, Gonorrhea, Impetigo, Leprosy, Legionellosis, Listeriosis, Lyme disease, Melioidosis, MRSA infection, Nocardiosis, Pertussis, Plague, Pneumococcal pneumonia, Psittacosis, Q fever, Rocky Mountain Spotted Fever, Salmonellosis, Scarlet fever, Shigellosis, Syphilis, Tetanus, Trachoma, Tuberculosis, Tularemia, Typhoid fever, Typhus, Urinary tract infections, Stafilococcus Aureus, Pseudomonans aeruginosa and Prpionobacterium acne.

5) The method of claim 1, wherein said infection or infectious disease is a viral infection selected from AIDS, AIDS related complex, Chickenpox, Common cold, Cytomegalovirus infection, Colorado tick fever, Ebola haemorrhagic fever, Dengue fever, Hand, foot and mouth disease, Hepatitis, Herpes simplex, Herpes zoster, HPV, Influenza, Lassa fever, Measles, Marburg haemorrhagic, fever, Infectious mononucleosis, Mumps, Poliomyelitis, Progressive multifocal leukencephalopathy, Rabies, Rubella, SARS, Smallpox, Viral encephalitis, Viral gastroenteritis, Viral meningitis, Viral pneumonia, West Nile disease and Yellow fever.

6) The method of claim 1, wherein said infection or infectious disease is a fungal infection selected from Aspergillosis, Blastomycosis, Candidiasis, Coccidioidomycosis, Cryptococcosis, Histoplasmosis, Tinea pedis, Candida albicans, Trichophyton mentagrophytes and Trichophyton rubrum.

7) The method of claim 1, wherein said infection or infectious disease is a parasitic infection selected from African trypanosomiasis, Amebiasis, Ascariasis, Babesiosis, Chagas disease, Clonorchiasis, Cryptosporidiosis, Cysticercosis, Diphyllobothriasis, Dracunculiasis, Echinococcosis, Enterobiasis, Fascioliasis, Fasciolopsiasis, Filariasis, Free-living amebic infection, Giardiasis, Gnathostomiasis, Hymenolepiasis, Isosporiasis, Leishmaniasis, Metagonimiasis, Myiasis, Onchocerciasis, Pediculosis, Scabies, Schistosomiasis, Taeniasis, Toxocariasis, Toxoplasmosis, Trichinellosis, Trichuriasis, Trichomoniasis, Trypanosomiasis and Trypanosoma.

8) The method of claim 1, wherein said infection or infectious disease is selected from onychomycosis, herpes virus, candida, T. mentagrophytes, gingivitis, aphthous ulcers, cornea ulcers, psoriasis, acne, atopic dermatitis, cellulites, folliculitis, boils, carbuncles, erysipelas, erythrasma, impetigo, paronychia, staphylococcal infection, cold sores (herpes simplex virus Type 1 and Type 2), HIV, moluscum contagiosum, chicken pox, measles, shingles, warts, candidiasis, athlete's foot (tinea pedis), jock itch (tinea cruris), ringworm (tinea corporis), face fungus (tinea faciei), tinea versicolor, fungal nail infections, fungal hair infections, lice, creeping eruption and scabies.

9) The method of claim 1, wherein said psoriasis is plaque, guttate, inverse, pustular or erythrodermic.

10) The method of claim 1, comprising topically applying said water to the skin or mucosa of said mammal.

11) The method of claim 1, comprising topically applying said water to the site of infection or lesion.

12) The method of claim 11, wherein said site of infection or lesion is selected from skin, mucosa, sinuses, eye, nose, ear, ear canal, mouth, lips, tongue, nails, elbows, knees, scalp, palms of hands, soles of feet, genitalia and vagina.

13) The method of claim 1, wherein the form of topical medication is selected from the group consisting of eye drop, ear drop, nose drop, mouth drop, moth sprays, nasal sprays, liquid formulation, paste, gels, creams and ointments.

14) A method of healing, repair or regeneration of a tissue in a mammal comprising topically administering to said tissue an electrolytic acid water comprising free chlorine, wherein:
a) greater than 95% of said free chlorine is present in the form of hypochlorous acid;
b) said water has a pH of from 0.5 to 5.0; and
c) said water has oxidation reduction potential (ORP) of greater than 1000 mV.

15) The method of claim 14, wherein said water is further characterized by a pH less than 3, free chlorine of from 40 to 70 ppm, and chloride of less than 200 ppm.

16) The method of claim 14, wherein said tissue is selected from the group consisting of skin, mucosa, sinuses, eye, nose, ear, ear canal, mouth, lips, tongue, nails, elbows, knees, scalp, palms of hands, soles of feet, genitalia and vagina.

17) The method of claim 14, comprising topically applying said water to the skin or mucosa of said tissue.

18) The method of claim 14, wherein said tissue has a wound.

19) The method of claim 14, wherein said tissue has a chronic wound.

20) The method of claim 19, wherein said chronic wound is a diabetic ulcer, venous ulcer, pressure ulcer, vasculitic ulcer or arterial ulcer.

21) The method of claim 14, wherein the form of topical medication is selected from the group consisting of eye drop, ear drop, nose drop, mouth drop, moth sprays, nasal sprays, liquid formulation, paste, gels, creams and ointments.

22) A method of washing, sanitizing, hydrating or storing a mammalian tissue or organ externalized from the body of said mammal, comprising contacting said tissue or organ with, or immersing said tissue or organ in, an electrolytic acid water comprising free chlorine, wherein:
a) greater than 95% of said free chlorine is present in the form of hypochlorous acid;
b) said water has a pH of from 0.5 to 5.0; and
c) said water has oxidation reduction potential (ORP) of greater than 1000 mV.

23) The method of claim 22, wherein said water is further characterized by a pH less than 3, free chlorine of from 40 to 70 ppm, and chloride of less than 200 ppm.

24) The method of claim 22, wherein said hydrating is rehydrating a dehydrated tissue or organ for implantation or transplantation.

25) The method of claim 22, wherein said tissue or organ is selected from heart, kidneys, liver, lungs, pancreas, intestine, thymus, bones, tendons (musculoskeletal grafts), cornea, skin, heart valves and veins.

26) The method of claim 22, comprising topically applying said water to the surface of said tissue or organ.

27) The method of claim 22, wherein said mammal is selected from human, dog, cat and horse.

28) The method of claim 22, wherein said mammal is a human.

29) The method of any one of claim 1, 14 or 22, wherein said water has a conductivity greater than 1,300 μS cm⁻¹.

30) The method of any one of claim 1, 14 or 22, wherein from 95% to 99.9% of said free chlorine is present in the form of hypochlorous acid.

31) The method of any one of claim 1, 14 or 22, wherein said water has a pH of from 1.0 to 4.0.

32) The method of any one of claim 1, 14 or 22, wherein:
a) from 95% to 99.9% of said free chlorine is present in the form of hypochlorous acid; and
b) said water has conductivity greater than 1,300 μS cm⁻¹.

33) The method of any one of claim 1, 14 or 22, wherein said water has a NMR half line width using ¹⁷O of from 42 to 60 Hz.

34) The method of any one of claim 1, 14 or 22, wherein said water has an anti-infection, or tissue healing, regeneration or repair effect.
